(19) United States
(12) Patent Application Publication
(10) Pub. No.: US 2024/0079105 A1
(43) Pub. Date: Mar. 7, 2024

(54) SYSTEMS AND METHODS FOR GENERATING HISTORY AND PHYSICAL EXAM NOTES

(71) Applicant: The UAB Research Foundation, Birmingham, AL (US)

(72) Inventor: Andrew Dennis Smith, Hoover, AL (US)

(21) Appl. No.: 18/460,320

(22) Filed: Sep. 1, 2023

Related U.S. Application Data
(60) Provisional application No. 63/403,557, filed on Sep. 2, 2022.

(51) Int. Cl. G16H 10/60; G16H 50/20

(52) U.S. Cl. CPC G16H 10/60; G16H 50/20

(57) ABSTRACT

A method for generating a medical record note comprises (i) accessing a corpus of medical documentation for a particular patient; (ii) receiving, at a user interface, chief complaint input indicating a chief complaint associated with the particular patient; (iii) utilizing (a) the chief complaint and (b) at least a portion of the corpus of medical documentation for the particular patient as input to a set of artificial intelligence (AI) modules, the set of AI modules being trained to generate medical record note output in response to medical documentation input; and (iv) generate a medical record note for the particular patient based on output of the set of AI modules.

### CROSS-REFERENCE TO RELATED APPLICATIONS

This application claims priority to U.S. Provisional Application No. 63/403,557, filed on Sep. 2, 2022, and entitled “SYSTEMS AND METHODS FOR GENERATING HISTORY AND PHYSICAL EXAM NOTES”, the entirety of which is incorporated herein by reference.

### BACKGROUND

A history and physical (H&P) exam note is a formal and complete assessment of the patient and a problem associated with a patient. A typical H&P describes the chief complaint, medical history, physical exam findings, laboratory/test findings, and an assessment and plan. H&P notes are included in electronic medical records (EMRs) for patients and are often completed prior to a procedure or change of care.

Many H&P exam notes build off of prior clinical notes and data in the EMR and are combined with patient questioning and physical exam findings. However, conventional techniques for generating an H&P exam note rely on manual text entry or basic speech recognition functionality. Accordingly, conventional techniques for H&P exam note generation are prone to errors, omissions, inefficiency, and poor standardization.

Therefore, there exists a substantial need for improved techniques for generating H&P exam notes.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 illustrates a conceptual representation of techniques for generating an H&P exam note, in accordance with implementations of the present disclosure.

FIGS. 2 and 3 illustrate example flow diagrams depicting acts associated with generating medical record notes, in accordance with implementations of the present disclosure.

FIG. 4 illustrates example aspects of a system that may comprise or implement one or more disclosed embodiments.

### DETAILED DESCRIPTION

Disclosed embodiments are generally directed to systems, methods, and devices for generating medical record notes, such as history and physical (H&P) exam notes. Although the present disclosure focuses, in at least some respects, on the generation of H&P exam notes, those skilled in the art will appreciate, in view of the present disclosure, that the principles discussed herein may be applied to the generation of other types of documents, such as discharge summaries.

While the detailed description may be separated into sections, the contents within each section are not intended to be self-contained descriptions and embodiments. Rather, the contents of each section within the detailed description are intended to be read and understood as a collective whole where elements of one section may pertain to and/or inform other sections. Accordingly, embodiments specifically disclosed within one section may also relate to and/or serve as additional and/or alternative embodiments in another section having the same and/or similar systems, modules, devices, methods, and/or terminology.

The embodiments disclosed herein will now be described by reference to some more detailed embodiments, with occasional reference to any applicable accompanying drawings. These embodiments may, however, be embodied in different forms and should not be construed as limited to the embodiments set forth herein. Rather, these embodiments are provided so that this disclosure will be thorough and complete, and will fully convey the scope of the embodiments to those skilled in the art.

### Examples of Technical Benefits, Improvements, and Practical Applications

Those skilled in the art will recognize, in view of the present disclosure, that at least some of the disclosed embodiments may be implemented to address various shortcomings associated with the generation of H&P exam notes. The following section outlines some example improvements and/or practical applications that may be achieved by implementing the disclosed embodiments. It will be appreciated, however, that the following are examples only and that the embodiments described herein are in no way limited to the example improvements discussed herein.

At least some embodiments disclosed herein include or are configured to perform various acts, such as accessing a corpus of medical documentation for a particular patient, providing at least a portion of the corpus as input to a set of artificial intelligence (AI) modules (which is trained to generate medical record note output in response to medical documentation input), and generating an H&P exam note for the particular patient based on output of the set of AI modules.

The set of AI module(s) may be trained at least in part utilizing training data that includes electronic medical record (EMR) data input and H&P exam note ground truth output. In some instances, the set of AI module(s) includes multiple AI modules operating in series, such as an extraction module to facilitate obtaining of relevant information from the corpus of medical documentation for the patient and an assessment module for analyzing the extracted information to generate an assessment and/or plan portion of the H&P exam note (e.g., at least partially in view of a chief complaint, which may be provided as an additional input). Utilizing AI to at least partially generate H&P exam notes in accordance with the present disclosure may reduce errors, omissions, and/or inefficiencies associated with conventional techniques for generating H&P exam notes.

In some instances, the H&P exam note is generated based on a chief complaint associated with the particular patient, which may be obtained via analysis of an EMR for the patient and/or based on user input (e.g., input provided by medical personnel) indicating the chief complaint. Utilizing a predefined chief complaint to guide the AI-based generation of an H&P exam note may reduce the search space of relevant content within the corpus of medical documentation for the patient, thereby improving computational speed and/or efficiency.

In some instances, an H&P exam note generated utilizing AI is presented to a user for modification and/or acceptance by the user (e.g., via user input provided by the user). For example, an H&P exam note generated utilizing AI may include recommended content, citations and/or links to portions of the corpus of medical documentation for recommended content, etc. A finalized H&P exam note may be generated based on the user input provided, and the finalized H&P exam note may be added to the corpus of medical documentation for the particular patient (e.g., added to the EMR for the patient). The finalized H&P exam note (and/or user input/input data used to finalize the H&P exam note) may advantageously be further utilized to refine and/or further train the set of AI module(s).

Having just described some of the various high-level features and benefits of the disclosed embodiments, attention will now be directed to the Figures, which illustrate various conceptual representations, architectures, methods, and supporting illustrations related to the disclosed embodiments.

### Example Techniques for Generating History and Physical Exam Notes

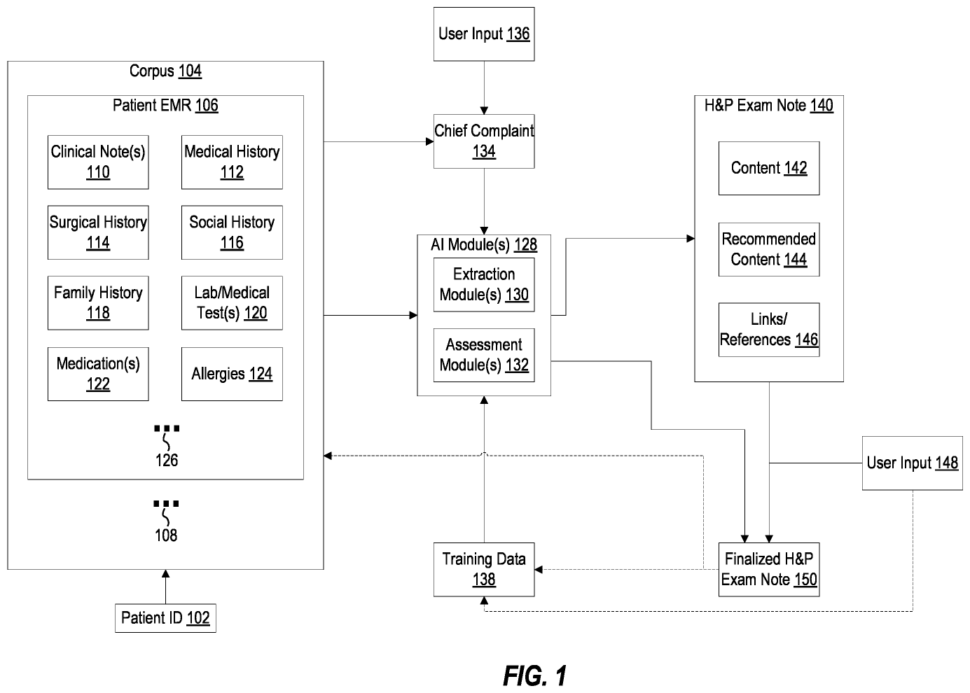

FIG. 1 depicts a conceptual representation of techniques for generating an H&P exam note for a particular patient. FIG. 1 illustrates a patient ID 102 associated with a particular patient for whom an H&P exam note will be generated according to the example(s) shown in FIG. 1. The patient ID 102 may take on any suitable form for identifying a particular patient for whom to generate an H&P exam note, such as a patient name, birthday, numerical identifier, combinations thereof, etc.

FIG. 1 also illustrates a corpus 104, which comprises a corpus of medical documentation. As will be described in more detail hereinafter, a system may utilize the corpus 104 to generate an H&P exam note for the particular patient. In some instances, the corpus 104 is identified (e.g., from a general database/corpus that includes medical documentation for multiple patients) based on the patient ID 102 and comprises medical documentation associated only with the particular patient (as indicated in FIG. 1 by the arrow extending from the patient ID 102 to the corpus 104).

The corpus 104 may comprise various types of medical documentation for the particular patient associated with the patient ID 102 (indicated in FIG. 1 by the ellipsis 108). By way of non-limiting example, FIG. 1 illustrates the corpus 104 as comprising a patient EMR 106 for the particular patient, which includes various medical information associated with the particular patient. FIG. 1 provides various non-limiting examples of patient information that may be included in a patient EMR 106, such as clinical note(s) 110, medical history 112, surgical history 114, social history 116, family history 118, lab/medical test(s) 120 (e.g., pathology findings, physical exams, systems reviews, diagnostic tests, radiology findings, etc.), medication(s) 122, allergies 124, medical record notes, and/or others (as indicated in FIG. 1 by the ellipsis 126). Such information may be obtained via interactions with medical practitioners, processing of sensor data (e.g., medical images) and/or samples obtained of/from the patient, etc. Different types of information represented within the patient EMR 106 may be stored/structured in different formats.

In some implementations, a system utilizes at least a portion of the corpus 104 to generate an H&P exam note. For example, FIG. 1 illustrates an arrow extending between the corpus 104 and AI module(s) 128, indicating that at least a portion of the corpus 104 may be utilized as input to AI module(s) 128 to facilitate generation of an H&P exam note 140 (indicated in FIG. 1 by an arrow extending from the AI module(s) 128 to the H&P exam note 140). In some implementations, the AI module(s) 128 comprise or utilize a language model (e.g., a large language model (LLM)) or natural language processing (NLP) model. Example language models include, but are not limited to, bidirectional encoder representations from transformers (BERT), generative pre-trained transformers (GPT-3, GPT 3.5, GPT 4, or other versions), Turing, PaLM, PaLM 2, Codex, Claude, Cohere, and/or others. The AI module(s) 128 may take on any suitable form, such as by comprising or utilizing hardware components and/or computer-executable instructions operable to carry out function blocks and/or processing layers configured in the form of, by way of non-limiting example, single-layer neural networks, feed forward neural networks, deep learning models, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, combinations thereof and/or others.

In some instances, the AI module(s) 128 comprise or implement one or more language models that is/are further refined via additional training to configure the language model(s) for particular uses/purposes. Various training techniques may be utilized to further refine the one or more language model(s), such as fully supervised, partially supervised, and/or unsupervised approaches. In one example, tagged training data 138 is utilized to train the AI module(s) 128 under a full supervision or weak supervision approach. Tagged training data may comprise domain-specific and/or enterprise-specific content that includes tags that link relevant medical record documentation content (e.g., information found in clinical notes, medical/lab tests, etc.) to aspects of an H&P exam note (e.g., chief complaint, history of present illness, medical history, surgical history, social history, family history, allergies, medications, review of systems, physical examination, diagnostic tests, assessment and plan, plan, etc.). Such tagged training data may be obtained in various ways. For example, existing H&P exam notes for patients may be tagged to link portions of the existing H&P exam notes to respective EMRs for patients. Such tagging may be performed manually (e.g., leveraging an enterprise's citation policy for creating H&P exam notes) and/or automatically (e.g., utilizing a separate AI module to determine the provenance of portions of existing H&P exam notes within EMRs for patients). In some instances, training data 138 for training the AI module(s) 128 may comprise EMR data input and H&P exam note ground truth output, which may be utilized to train and/or refine the AI module(s) 128 in an at least partially supervised manner.

FIG. 1 also illustrates a conceptual representation of a chief complaint 134, which may be used as an input to the AI module(s) 128 to facilitate generation of the H&P exam note 140. In some instances, the chief complaint describes a bodily problem, condition, and/or symptom experienced by the user that the H&P exam note 140 is configured to address. In this regard, the H&P exam note 140 may be generated based on the chief complaint 134 and/or may include the chief complaint 134. The chief complaint 134 may be obtained based on user input 136 (e.g., input by medical personnel based on a patient-provided description of the problem, input provided by the patient directly, etc.), content of the corpus 104 (e.g., a complaint present in the patient EMR 106 that is associated with a recent timepoint), and/or other sources (e.g., sensor data, etc.). For example, a system may obtain one or more recent patient complaints from the patient EMR 106 and may present them to a user for selection at a user interface. The user may provide user input 136 selecting one of the presented candidate complaints or defining a different complaint (e.g., based on interactions with the patient). The selected or defined complaint may be utilized as the chief complaint 134 (e.g., utilized as user input to the AI module(s) 128) and form a basis for generating at least part of the H&P exam note 140. FIG. 1 conceptually represents the foregoing with arrows extending from the corpus 104 and user input 136 to the chief complaint, and with an arrow extending from the chief complaint 134 to the AI module(s) 128. In some implementations, a system utilizes the chief complaint 134 to determine medical documentation information to include in the corpus 104 for generating the H&P exam note 140.

As illustrated in FIG. 1, the AI module(s) 128 may comprise one or more separate AI modules, such as extraction module(s) 130 and assessment module(s) 132. In some instances, extraction module(s) 130 and the assessment module(s) 132 are configured to generate different portions of the H&P exam note 140. For example, the extraction module(s) 130 may be configured to obtain relevant portions of the patient EMR 106 (e.g., relevant portions of clinical note(s) 110, lab/medical test(s) 120, etc.) to generate a history and status portion of the H&P exam note 140, whereas the assessment module(s) 132 may be configured to generate an assessment and plan portion of the H&P exam note 140. The history and status portion of the H&P exam note 140 may comprise various subsections, such as, by way of non-limiting example, history of present illness, surgical history, medical history, social history, family history, allergies, medications, review of systems, physical examination, pertinent diagnostic tests, problem list, and/or others. The assessment and plan portion of the H&P exam note 140 may similarly comprise various subsections, such as, by way of non-limiting example, an assessment section, a recommendation section, and/or a plan section.

The extraction module(s) 130 and the assessment module(s) 132 may be configured to operate in series to generate the H&P exam note 140. For instance, history and status H&P exam note content obtained/generated by the extraction module(s) 130 may be utilized as an input to the assessment module(s) 132 to generate the assessment and plan portion of the H&P exam note 140. The extraction module(s) 130 and the assessment module(s) 132 may comprise any suitable type of AI module(s) (e.g., NLP models).

In some instances, the extraction module(s) 130 is/are trained utilizing training data 138 as discussed hereinabove (e.g., dataset(s) including tags that link portions of existing H&P exam notes to existing patient EMRs), and the assessment module(s) 132 is/are trained utilizing training data that includes history and status portions of existing H&P exam notes as training input and assessment and plan portions of existing H&P exam notes as ground truth output.

One will appreciate, in view of the present disclosure, that the AI module(s) 128 may comprise or operate in conjunction with other types of functions to facilitate generation of the H&P exam note 140. For example, at least some portions of an H&P exam note may be generated utilizing rules-based functions (e.g., an allergies section or physical examination section may be obtained utilizing a function that at least partially relies on keyword matching functionality or other matching logic to identify relevant sections of the patient EMR 106 from which to obtain allergy and/or physical examination information).

Accordingly, a system may utilize output of the AI module(s) 128 to generate the H&P exam note 140. In the example shown in FIG. 1, the H&P exam note 140 may comprise content 142, recommended content 144, and/or links/references 146. The content 142 and/or the recommended content 144 may comprise one or more aspects of the history and status sections and/or the assessment and plan sections discussed hereinabove. The links/references 146 may indicate and/or link to portions of the corpus 104 that the AI module(s) 128 identified as bases for the content 142 and/or recommended content 144.

Content 142 and/or recommended content 144 may be presented at a user interface for modification and/or acceptance by a human user. For example, recommended content 144 may comprise recommended textual content usable within a “history of present illness” section of the H&P exam note 140 (e.g., a description of the history of the present illness in prose, or provided in key point format, and/or multiple alternative descriptions/key points for selection by the user), and the links/references 146 may include references to portions of the EMR 106 that provide a basis for the textual content. Such recommended content 144 and/or links/references may be presented to a human user at as user interface to allow the user to modify and/or accept the recommended content 144 (indicated in FIG. 1 by user input 148) to form the “history of present illness” section of the H&P exam note 140. The user input 148 148 modifying or accepting portions of the H&P exam note 140 may be used to generate a finalized H&P exam note 150, as indicated in FIG. 1 by the arrows extending from the H&P exam note 140 and the user input 148 toward the finalized H&P exam note 150.

In some instances, user input 148 modifying the H&P exam note 140 may trigger automatic modification to the H&P exam note 140. In one example, user input 148 indicating that a portion of the patient EMR 106 is not relevant to content 142 of the history and status section of the H&P exam note 140 may cause automatic modification to the history and status section of the H&P exam note 140 (and/or to the recommended content 144). Such automatic modification may be facilitated via the AI module(s) 128 (e.g., the extraction module(s) 130). In another example, user input 148 modifying a history and status section of the H&P exam note 140 may cause automatic modification to the assessment plan portion of the H&P exam note 140. Such automatic modification may be obtained via the AI module(s) 128 (e.g., assessment module(s) 132). Such functionality is conceptually represented in FIG. 1 by the arrow extending from the AI module(s) 128 to the finalized H&P exam note 150.

A finalized H&P exam note 150 generated in accordance with the principles disclosed hereinabove may be used to facilitate addressing of the chief complaint 134 of the patient by medical practitioners. Furthermore, in some instances, the finalized H&P exam note 150 may be added to the corpus 104 for the particular patient (e.g., added to the patient EMR 106) such that the finalized H&P exam note 150 may be parsed for generating subsequent medical record notes (e.g., subsequent H&P exam notes) (represented in FIG. 1 by the dashed arrow extending from the finalized H&P exam note 150 to the corpus 104). Still furthermore, in some instances, the finalized H&P exam note 150 may be utilized as training data to further train/refine the AI module(s) 128 in accordance with the training methodologies discussed hereinabove (represented in FIG. 1 by the dashed arrow extending from the finalized H&P exam note 150 to the training data 138). Additionally, or alternatively, the training data 138 for training the AI module(s) 128 may be at least partially based on user input 148 modifying or accepting the H&P exam note 140 to generate the finalized H&P exam note 150 (indicated in FIG. 1 by the dashed arrow extending from the user input 148 to the training data 138).

### Example Method(s)

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

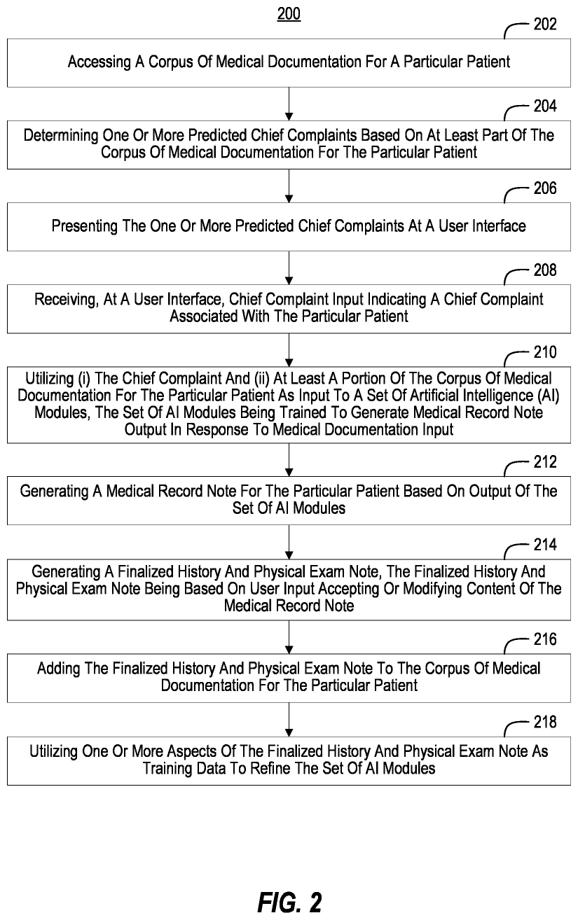

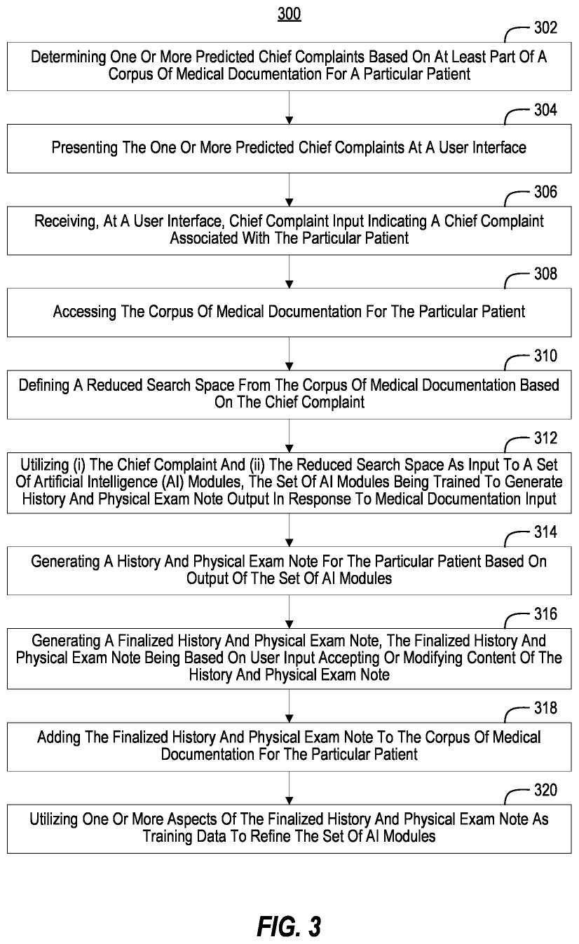

FIGS. 2 and 3 illustrate example flow diagrams 200 and 300, respectively, depicting acts associated with generating a medical record note, in accordance with implementations of the present disclosure.

Act 202 of flow diagram 200 of FIG. 2 includes accessing a corpus of medical documentation for a particular patient. The patient may comprise a medical patient. The corpus of medical documentation may comprise electronic medical record (EMR) data associated with the particular patient. For instance, EMR data for the particular patient may be identified based on a patient identifier (e.g., patient name, birthday, numerical identifier, etc.) provided to or accessed by a system.

Act 204 of flow diagram 200 includes determining one or more predicted chief complaints based on at least part of the corpus of medical documentation for the particular patient. A predicted chief complaint may comprise a predicted bodily problem, condition, and/or symptom experienced by the particular patient. For instance, a system may parse or process the corpus of medical documentation for the particular patient to determine the predicted chief complaint(s). Predicted chief complaints may be selected based on various factors, such as temporal recency, previous diagnoses, conditions, test results, etc.

Act 206 of flow diagram 200 includes presenting the one or more predicted chief complaints at a user interface. The predicted chief complaint(s) may be presented in any suitable format, such as visual, audible, tactile, or other formats. The user interface used to present the predicted chief complaint(s) may take on any suitable form (e.g., a system 400 as described hereinafter).

Act 208 of flow diagram 200 includes receiving, at a user interface, chief complaint input indicating a chief complaint associated with the particular patient. In some instances, the chief complaint input is provided at the same user interface where the predicted chief complaint(s) are presented in accordance with act 206 (when act 206 is performed in combination with act 208). In instances where act 206 is performed, the chief complaint input may comprise user input that (i) selects the chief complaint from among the one or more predicted chief complaints, (ii) defines the chief complaint by modifying at least one of the one or more predicted chief complaints, or (iii) rejects the one or more predicted chief complaints and defines the chief complaint. The user input may be provided in any suitable form, such as voice input, device input (e.g., typing, clicking, touching, etc.), gesture input, and/or others. In instances where act 206 is not performed, the chief complaint input may comprise user input that defines the chief complaint. For instance, a medical practitioner may enter the chief complaint (e.g., via voice or other input modality) at a user interface after consultation with and/or examination of the particular patient (or the particular patient may input the chief complaint themselves for review/finalization by a medical practitioner).

Act 210 of flow diagram 200 includes utilizing (i) the chief complaint and (ii) at least a portion of the corpus of medical documentation for the particular patient as input to a set of artificial intelligence (AI) modules, the set of AI modules being trained to generate medical record note output in response to medical documentation input. One will appreciate that various pre-processing and/or formatting may be performed on the chief complaint and/or the portion of the corpus of medical documentation prior to processing by the set of AI modules (e.g., lemmatization, lowercase conversion, document conversion, document reformatting, optical character recognition). In some instances, the portion of the corpus of medical documentation utilized as input to the set of AI modules is selected based on the chief complaint. For instance, portions of a patient's EMR may be selected for or omitted from processing by the set of AI modules based on relevance to the chief complaint. As described hereinabove, the set of AI modules may comprise various types of processing blocks/modules and may take on any suitable form, such as one or more language models. The set of AI modules may be trained and/or fine-tuned using training data that includes (i) EMR data and chief complaint data input and (ii) history and physical exam note ground truth output. In some implementations, the set of AI modules comprises at least a first AI module and a second AI module configured to operate in series. The first AI module may be configured to generate a history and status portion of the history and physical exam note, and the second AI module may be configured to generate an assessment and plan portion of the history and physical exam note based at least in part on output of the first AI module.

Act 212 of flow diagram 200 includes generating a medical record note for the particular patient based on output of the set of AI modules. As indicated above, in at least some implementations, the medical record note comprises a history and physical exam note, which can include various sections, such as, by way of non-limiting example, chief complaint, history and status, assessment and plan, etc. The history and status section may comprise various subsections, such as, by way of non-limiting example, history of present illness, surgical history, medical history, social history, family history, allergies, medications, review of systems, physical examination, pertinent diagnostic tests, problem list, and/or others. The assessment and plan section may comprise various subsections, such as, by way of non-limiting example, assessment, recommendation, plan, etc.

Act 214 of flow diagram 200 includes generating a finalized history and physical exam note, the finalized history and physical exam note being based on user input accepting or modifying content of the medical record note. The user input may be provided at any suitable user interface. For instance, the history and physical exam note (e.g., medical record note) generated according to act 212 may be presented at a user interface (e.g., in the form of a text document in a word processor, or in another format such as a guided workflow) while prompting the user to accept, modify, or reject portions of the history and physical exam note to form the finalized history and physical exam note. The finalized history and physical exam note may be utilized for patient care purposes.

Act 216 of flow diagram 200 includes adding the finalized history and physical exam note to the corpus of medical documentation for the particular patient. In this way, the finalized history and physical exam note may be accessible as part of the corpus of medical documentation for the particular patient to generate future history and physical exam notes for the particular patient, according to the techniques described herein.

Act 218 of flow diagram 200 includes utilizing one or more aspects of the finalized history and physical exam note as training data to refine the set of AI modules. For instance, further backpropagation or other modification to parameters and/or hyperparameters of one or more components of the set of AI modules may be performed based on the user-driven changes made to the history and physical exam note to obtain the finalized history and physical exam note.

Act 302 of flow diagram 300 of FIG. 3 includes determining one or more predicted chief complaints based on at least part of a corpus of medical documentation for a particular patient. The patient may comprise a medical patient. The corpus of medical documentation may comprise electronic medical record (EMR) data associated with the particular patient. For instance, EMR data for the particular patient may be identified based on a patient identifier (e.g., patient name, birthday, numerical identifier, etc.) provided to or accessed by a system. A predicted chief complaint may comprise a predicted bodily problem, condition, and/or symptom experienced by the particular patient. For instance, a system may parse or process the corpus of medical documentation for the particular patient to determine the predicted chief complaint(s). Predicted chief complaints may be selected based on various factors, such as temporal recency, previous diagnoses, conditions, test results, etc.

Act 304 of flow diagram 300 includes presenting the one or more predicted chief complaints at a user interface. The predicted chief complaint(s) may be presented in any suitable format, such as visual, audible, tactile, or other formats. The user interface used to present the predicted chief complaint(s) may take on any suitable form (e.g., a system 400 as described hereinafter).

Act 306 of flow diagram 300 includes receiving, at a user interface, chief complaint input indicating a chief complaint associated with the particular patient. In some instances, the chief complaint input is provided at the same user interface where the predicted chief complaint(s) are presented in accordance with act 206 (when act 206 is performed in combination with act 208). In instances where act 206 is performed, the chief complaint input may comprise user input that (i) selects the chief complaint from among the one or more predicted chief complaints, (ii) defines the chief complaint by modifying at least one of the one or more predicted chief complaints, or (iii) rejects the one or more predicted chief complaints and defines the chief complaint. The user input may be provided in any suitable form, such as voice input, device input (e.g., typing, clicking, touching, etc.), gesture input, and/or others. In instances where act 206 is not performed, the chief complaint input may comprise user input that defines the chief complaint. For instance, a medical practitioner may enter the chief complaint (e.g., via voice or other input modality) at a user interface after consultation with and/or examination of the particular patient (or the particular patient may input the chief complaint themselves for review/finalization by a medical practitioner).

Act 308 of flow diagram 300 includes accessing the corpus of medical documentation for the particular patient. As noted above, the corpus of medical documentation may comprise electronic medical record (EMR) data associated with the particular patient.

Act 310 of flow diagram 300 includes defining a reduced search space from the corpus of medical documentation based on the chief complaint. For instance, the reduced search space may be defined by selecting or omitting portions of a patient's EMR based on relevance to the chief complaint. Various techniques and/or machine learning modules may be employed to determine relevance of portions of the patient's EMR to the chief complaint, such as topic analysis modules, categorization modules (e.g., text classification/grouping modules), similarity analysis modules (for representing text with vectors in vector space and comparing vectors to determine similarity), and/or others.

Act 312 of flow diagram 300 includes utilizing (i) the chief complaint and (ii) the reduced search space as input to a set of artificial intelligence (AI) modules, the set of AI modules being trained to generate history and physical exam note output in response to medical documentation input. One will appreciate that various pre-processing and/or formatting may be performed on the chief complaint and/or the reduced search space prior to processing by the set of AI modules (e.g., lemmatization, lowercase conversion, document conversion, document reformatting, optical character recognition). As described hereinabove, the set of AI modules may comprise various types of processing blocks/modules and may take on any suitable form, such as one or more language models. The set of AI modules may be trained and/or fine-tuned using training data that includes (i) EMR data and chief complaint data input and (ii) history and physical exam note ground truth output. In some implementations, the set of AI modules comprises at least a first AI module and a second AI module configured to operate in series. The first AI module may be configured to generate a history and status portion of the history and physical exam note, and the second AI module may be configured to generate an assessment and plan portion of the history and physical exam note based at least in part on output of the first AI module.

Act 314 of flow diagram 300 includes generating a history and physical exam note for the particular patient based on output of the set of AI modules. In some implementations, the history and physical exam note can include various sections, such as, by way of non-limiting example, chief complaint, history and status, assessment and plan, etc. The history and status section may comprise various subsections, such as, by way of non-limiting example, history of present illness, surgical history, medical history, social history, family history, allergies, medications, review of systems, physical examination, pertinent diagnostic tests, problem list, and/or others. The assessment and plan section may comprise various subsections, such as, by way of non-limiting example, assessment, recommendation, plan, etc.

Act 316 of flow diagram 300 includes generating a finalized history and physical exam note, the finalized history and physical exam note being based on user input accepting or modifying content of the history and physical exam note. The user input may be provided at any suitable user interface. For instance, the history and physical exam note generated according to act 314 may be presented at a user interface (e.g., in the form of a text document in a word processor, or in another format such as a guided workflow) while prompting the user to accept, modify, or reject portions of the history and physical exam note to form the finalized history and physical exam note. The finalized history and physical exam note may be utilized for patient care purposes.

Act 318 of flow diagram 300 includes adding the finalized history and physical exam note to the corpus of medical documentation for the particular patient. In this way, the finalized history and physical exam note may be accessible as part of the corpus of medical documentation for the particular patient to generate future history and physical exam notes for the particular patient, according to the techniques described herein.

Act 320 of flow diagram 300 includes utilizing one or more aspects of the finalized history and physical exam note as training data to refine the set of AI modules. For instance, further backpropagation or other modification to parameters and/or hyperparameters of one or more components of the set of AI modules may be performed based on the user-driven changes made to the history and physical exam note to obtain the finalized history and physical exam note.

### Additional Details Related to Implementing the Disclosed Embodiments

The principles disclosed herein may be implemented in various formats. For example, the various techniques discussed herein may be performed as a method that includes various acts for achieving particular results or benefits. In some instances, the techniques discussed herein are represented in computer-executable instructions that may be stored on one or more computer-readable recording media. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

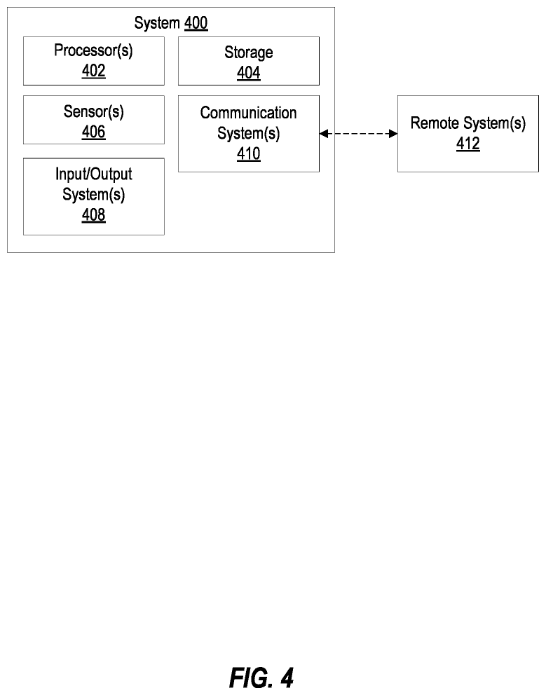

FIG. 4 illustrates example components of a system 400 that may comprise or implement aspects of one or more disclosed embodiments. For example, FIG. 4 illustrates an implementation in which the system 400 includes processor(s) 402, storage 404, sensor(s) 406, I/O system(s) 408, and communication system(s) 410. Although FIG. 4 illustrates a system 400 as including particular components, one will appreciate, in view of the present disclosure, that a system 400 may comprise any number of additional or alternative components.

The processor(s) 402 may comprise one or more sets of electronic circuitries that include any number of logic units, registers, and/or control units to facilitate the execution of computer-readable instructions (e.g., instructions that form a computer program). Such computer-readable instructions may be stored within storage 404. The storage 404 may comprise physical system memory (e.g., computer-readable recording media) and may be volatile, non-volatile, or some combination thereof. Furthermore, storage 404 may comprise local storage, remote storage (e.g., accessible via communication system(s) 410 or otherwise), or some combination thereof. Additional details related to processors (e.g., processor(s) 402) and computer storage media (e.g., storage 404) will be provided hereinafter.

As will be described in more detail, the processor(s) 402 may be configured to execute instructions stored within storage 404 to perform certain actions. In some instances, the actions may rely at least in part on communication system(s) 410 for receiving data from remote system(s) 412, which may include, for example, separate systems or computing devices, sensors, and/or others. The communications system(s) 410 may comprise any combination of software or hardware components that are operable to facilitate communication between on-system components/devices and/or with off-system components/devices. For example, the communications system(s) 410 may comprise ports, buses, or other physical connection apparatuses for communicating with other devices/components. Additionally, or alternatively, the communications system(s) 410 may comprise systems/components operable to communicate wirelessly with external systems and/or devices through any suitable communication channel(s), such as, by way of non-limiting example, Bluetooth, ultra-wideband, WLAN, infrared communication, and/or others.

FIG. 4 illustrates that a system 400 may comprise or be in communication with sensor(s) 406. Sensor(s) 406 may comprise any device for capturing or measuring data representative of perceivable phenomenon. By way of non-limiting example, the sensor(s) 406 may comprise one or more image sensors, microphones, thermometers, barometers, magnetometers, accelerometers, gyroscopes, and/or others.

Furthermore, FIG. 4 illustrates that a system 400 may comprise or be in communication with I/O system(s) 408. I/O system(s) 408 may include any type of input or output device such as, by way of non-limiting example, a display, a touch screen, a mouse, a keyboard, a controller, and/or others, without limitation.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media are computer-readable hardware storage devices, such as RAM, ROM, EEPROM, CD-ROM, solid state drives (“SSD”) that are based on RAM, Flash memory, phase-change memory (“PCM”), or other types of computer-readable recording media, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that at least some aspects of the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, at least some of the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A system for generating a medical record note, the system comprising:
one or more processors; and
one or more computer-readable recording media that store instructions that are executable by the one or more processors to configure the system to:
access a corpus of medical documentation for a particular patient;
receive, at a user interface, chief complaint input indicating a chief complaint associated with the particular patient;
utilize (i) the chief complaint and (ii) at least a portion of the corpus of medical documentation for the particular patient as input to a set of artificial intelligence (AI) modules, the set of AI modules being trained to generate medical record note output in response to medical documentation input; and
generate a medical record note for the particular patient based on output of the set of AI modules.

2. The system of claim 1, wherein the instructions are executable by the one or more processors to further configure the system to determine one or more predicted chief complaints based on at least part of the corpus of medical documentation for the particular patient.

3. The system of claim 2, wherein the one or more predicted chief complaints are determined based at least in part on temporal recency.

4. The system of claim 2, wherein the instructions are executable by the one or more processors to further configure the system to present the one or more predicted chief complaints at the user interface, and wherein the chief complaint input comprises user input that (i) selects the chief complaint from among the one or more predicted chief complaints, (ii) defines the chief complaint by modifying at least one of the one or more predicted chief complaints, or (iii) rejects the one or more predicted chief complaints and defines the chief complaint.

5. The system of claim 1, wherein the portion of the corpus of medical documentation for the particular patient utilized as input to the set of AI modules is selected based on the chief complaint.

6. The system of claim 1, wherein the corpus of medical documentation for the particular patient comprises an electronic medical record (EMR) for the particular patient.

7. The system of claim 6, wherein the medical record note comprises a history and physical exam note.

8. The system of claim 7, wherein the set of AI modules is trained using training data comprising EMR data and chief complaint data input and history and physical exam note ground truth output.

9. The system of claim 7, wherein the set of AI modules comprises at least a first AI module and a second AI module configured to operate in series, wherein the first AI module is configured to generate a history and status portion of the history and physical exam note, and wherein the second AI module is configured to generate an assessment and plan portion of the history and physical exam note based at least in part on output of the first AI module.

10. The system of claim 7, wherein the instructions are executable by the one or more processors to further configure the system to:
generate a finalized history and physical exam note, the finalized history and physical exam note being based on user input accepting or modifying content of the history and physical exam note.

11. The system of claim 10, wherein the instructions are executable by the one or more processors to further configure the system to:
add the finalized history and physical exam note to the corpus of medical documentation for the particular patient.

12. The system of claim 10, wherein the instructions are executable by the one or more processors to further configure the system to:
utilize one or more aspects of the finalized history and physical exam note as training data to refine the set of AI modules.

13. A system for generating a medical record note, the system comprising:
one or more processors; and
one or more computer-readable recording media that store instructions that are executable by the one or more processors to configure the system to:
receive, at a user interface, chief complaint input indicating a chief complaint associated with a particular patient;
access a corpus of medical documentation for the particular patient;
define a reduced search space from the corpus of medical documentation based on the chief complaint;
utilize (i) the chief complaint and (ii) the reduced search space as input to a set of artificial intelligence (AI) modules, the set of AI modules being trained to generate history and physical exam note output in response to medical documentation input; and
generate a history and physical exam note for the particular patient based on output of the set of AI modules.

14. The system of claim 13, wherein the instructions are executable by the one or more processors to further configure the system to determine one or more predicted chief complaints based on at least part of the corpus of medical documentation for the particular patient.

15. The system of claim 14, wherein the one or more predicted chief complaints are determined based at least in part on temporal recency.

16. The system of claim 14, wherein the instructions are executable by the one or more processors to further configure the system to present the one or more predicted chief complaints at the user interface, and wherein the chief complaint input comprises user input that (i) selects the chief complaint from among the one or more predicted chief complaints, (ii) defines the chief complaint by modifying at least one of the one or more predicted chief complaints, or (iii) rejects the one or more predicted chief complaints and defines the chief complaint.

17. The system of claim 13, wherein the corpus of medical documentation for the particular patient comprises an electronic medical record (EMR) for the particular patient, and wherein the set of AI modules is trained using training data comprising EMR data and chief complaint data input and history and physical exam note ground truth output.

18. The system of claim 13, wherein the set of AI modules comprises at least a first AI module and a second AI module configured to operate in series, wherein the first AI module is configured to generate a history and status portion of the history and physical exam note, and wherein the second AI module is configured to generate an assessment and plan portion of the history and physical exam note based at least in part on output of the first AI module.

19. The system of claim 13, wherein the instructions are executable by the one or more processors to further configure the system to:
generate a finalized history and physical exam note, the finalized history and physical exam note being based on user input accepting or modifying content of the history and physical exam note;
add the finalized history and physical exam note to the corpus of medical documentation for the particular patient; and
utilize one or more aspects of the finalized history and physical exam note as training data to refine the set of AI modules.

20. A method for generating a medical record note, the method comprising:
accessing a corpus of medical documentation for a particular patient;
utilizing at least a portion of the corpus of medical documentation for the particular patient as input to a set of artificial intelligence (AI) modules, the set of AI modules being trained to generate medical record note output in response to medical documentation input, wherein the set of AI modules comprises at least a first AI module and a second AI module configured to operate in series, wherein the first AI module is configured to generate a history and/or status portion of the medical record note output, and wherein the second AI module is configured to generate an assessment and/or plan portion of the medical record note output based at least in part on output of the first AI module; and
generating a medical record note for the particular patient based on output of the set of AI modules.
